(19) United States
(12) Patent Application Publication
(10) Pub. No.: US 2026/0092247 A1
(43) Pub. Date: Apr. 2, 2026

(54) Tuning Resonant Sensor Dynamic Response to Specific Cell Growth Rates by Adjusting Kerf Size

(71) Applicant: Skroot Laboratory, Inc., Ames, IA (US)

(72) Inventors: Nigel F. Reuel, Ames, IA (US); Samuel Rothstein, Ames, IA (US); Subhanwit Roy, Ames, IA (US); Aidan Shervheim, Ames, IA (US)

(73) Assignee: Skroot Laboratory, Inc., Ames, IA (US)

(21) Appl. No.: 19/341,419

(22) Filed: Sep. 26, 2025

Related U.S. Application Data
(60) Provisional application No. 63/701,272, filed on Sep. 30, 2024.

(51) Int. Cl. C12M 1/34; C12M 1/00

(52) U.S. Cl. CPC C12M 41/46; C12M 23/34

(57) ABSTRACT

An apparatus for monitoring cell cultures includes a resonant sensor having a cell transduction membrane for contact with cell culture media, a biocompatible sheet overlaid by the cell transduction membrane, a resonant sheet defining a coil pattern, the resonant sheet overlayed by the biocompatible sheet, a plurality of channels formed in the biocompatible sheet, the plurality channels of having a kerf. The kerf for the plurality of channels is selected such that the resonant sensor is configured to capture exponential phase of cell growth including late exponential cell growth and further capture stationary phases of cell growth for the cell cultures.

### PRIORITY STATEMENT

This application claims priority to U.S. Provisional Patent Application No. 63/701,272, filed Sep. 30, 2024, entitled Tuning Resonant Sensor Dynamic Response to Specific Cell Growth Rates by Adjusting Kerf Size, hereby incorporated by reference in its entirety.

### FIELD OF THE INVENTION

The present invention relates to monitoring of cell cultures. More particularly, but not exclusively, the present invention relates to monitoring cell growth with enhanced resonant sensors where channel width is modulated.

### BACKGROUND OF THE ART

The existing solutions for tracking growth in cell cultures varies depending on the cell culture application, but they can be summarized to three primary categories: optical measurements, cell counting/imaging, and biomass probes.

Optical based methods are commonly used in cell cultures to relate the turbidity of the cell culture to an estimated cell density. This light absorbance (optical density) or transmittance, when tracked rigorously and accurately, can mimic a pattern to traditional cell growth curves. This method utilizes Beer's Law to relate absorbance and concentration of cells in a sample. Generally, this relationship is accurate below an absorbance of 1, which for most cell cultures captures a small fraction of the cell growth curve. Optical density measurements beyond an absorbance of 1 necessitate dilution of the culture or sample, which can lead to errors or inconsistencies. Using optical cell density or a similar reflectance/transmission light strategy to track cell growth may be conducted continuously or intermittently. In the case of continuous optical cell density tracking, the vessel housing the cell culture needs to be optically clear for accurate readings (such as the OPTURA product line from Aber Instruments, Ltd.). The majority of these optically clear vessels are limited to clear glass vessels and plastic vessels, thereby excluding opaque cell culture vessels. Intermittent optical density measurements requires sampling of a well-mixed culture, generally by a technician, to analyze in a spectrophotometer.

Another method for tracking growth of a cell culture is to image and count the cells in a sample. Cell counting has similar risks to intermittent optical density analysis in that it requires a technician to remove a representative sample (well mixed system) of the culture which risks contamination, is labor intensive, and only gives intermittent reference points to cell culture growth. Moreover, imaging and automated cell counting software often have issues in accurately counting cells in a sample when the cells begin to clump together, like in the case of many mammalian cell cultures.

Biomass probes are another method for evaluating cell growth in a culture. Many of these biomass probes use an electric field to evaluate capacitance changes in the culture vessel to evaluate cell growth. This method of evaluating cell growth works best in large (>5 L) vessels that are well mixed. Due to the size of the probes used for tracking biomass, they are generally not used in smaller cell culture vessels that are commonly used in Research and Development departments or for manufacture of personalized cell therapies. Biomass probes further rely on the polarization of live cells to result in changes in the probe reading.

Therefore, there are limitations with all of these current approaches. Although it is recognized that resonant sensors can be used to monitor the growth of cells directly via changes in permittivity in the close vicinity of the sensors there may be difficulties in implementing such an approach, including limitations in the sensitivity.

Thus, different approaches for tracking growth in cell cultures have various issues. What is needed are new and innovative methods and apparatus for tracking growth in cell cultures which avoid and/or address these issues and improve over the state of the art.

### SUMMARY

Therefore, it is a primary object, feature, or advantage to improve over the state of the art.

It is a further object, feature, or advantage to enhance resonant sensors used in monitoring cell growths.

It is a still further object, feature, or advantage to modulate channel width in resonant sensors to tailor dynamic response to be proportionate with cell growth rates.

Another object, feature, or advantage is to provide continuous and real-time monitoring of cell cultures without the need to periodically sample the cell culture.

One or more of these and/or other objects, features, or advantages will become apparent from the specification and claims that follow. No single embodiment need exhibit each and every object, feature, or advantage as different embodiments may have different objects, features, or advantages. Thus, the present invention is not to be limited to or by any of these objects, features, or advantages.

According to one aspect, a resonant sensor may be used to monitor the growth of cells via changes in permittivity caused by the cells directly or to the growth media. The sensor may be a single-use, metabolite absorbing, resonant transducer with a responsive membrane that increases the signal to noise of the sensor when monitoring the growth of cells (bacteria, yeast, and mammalian cells). The sensor may include a biocompatible sheet with engineered voids that can be tailored to alter sensor dynamic response to be proportionate with cell growth rates. This sensor may enable continuous, real-time monitoring of cell cultures without the need to periodically sample the cell culture. The modulation of the channel width, i.e., kerf, allows for tailoring sensor gain and responsiveness to cell culture growth. Tailoring the kerf of the plastic channels in the resonant transducer sensor to best match cell growth data enables users to establish a direct relationship between sensor response to cell growth and sampled cell growth. The rapid assessment of sensor response may then be used as a proxy for estimating actual cell growth without the necessity of sampling cell cultures.

According to another aspect, an apparatus for monitoring cell cultures includes a resonant sensor having a cell transduction membrane for contact with cell culture media, a biocompatible sheet overlaid by the cell transduction membrane, a resonant sheet defining a coil pattern, the resonant sheet overlayed by the biocompatible sheet, a plurality of channels formed in the biocompatible sheet, the plurality channels of having a kerf. The kerf for the plurality of channels is selected such that the resonant sensor is configured to capture exponential phase of cell growth including late exponential cell growth and further capture stationary phases of cell growth for the cell cultures.

According to another aspect, a method of monitoring cell cultures is provided. The method includes selecting a resonant sensor, the resonant sensor having a cell transduction membrane for contact with cell culture media, a biocompatible sheet overlaid by the cell transduction membrane, a resonant sheet defining a coil pattern, the resonant sheet overlayed by the biocompatible sheet, a plurality of channels formed in the biocompatible sheet, the plurality channels of having a kerf, and wherein the kerf for the plurality of channels is selected such that the resonant sensor is configured to capture exponential phase of cell growth including late exponential cell growth and further capture stationary phases of cell growth for the cell cultures. The method may further include securing the resonant sensor to an inner wall of a vessel containing the cell culture media and during the cell culture, monitoring cell growth using the resonant sensor. The resonant sensor may be a single-use, metabolite absorbing, resonant sensor. The method may further include positioning a reader antenna in operative communication with the resonant sensor where the resonant sensor is positioned on the inner wall of a vessel during the cell culture.

According to another aspect, a method includes providing a resonant sensor, the resonant sensor includes a cell transduction membrane for contact with cell culture media, a biocompatible sheet overlaid by the cell transduction membrane, a resonant sheet defining a coil pattern, the resonant sheet overlayed by the biocompatible sheet, and a plurality of channels formed in the biocompatible sheet, the plurality channels of having a kerf. The method further includes tuning a dynamic response of the resonant sensor to a specific cell growth rate by adjusting size of the kerf.

### BRIEF DESCRIPTION OF THE DRAWINGS

FIG. 1 is a schematic illustration of an example environment in which a resonant sensor is used within a cell culture device. The figure shows a vessel containing cell culture media and a resonant sensor positioned against the inner surface of the vessel, with an external reader in operative communication with the resonant sensor.

FIG. 2 is a cross-sectional view of an example resonant sensor. The resonant sensor is comprised of multiple layers, including a membrane transduction layer (which may be a polyacrylate adhesive film), a biocompatible sheet which may be a plastic sheet such as a polyethylene terephthalate (PET) sheet with channels, and a resonator sheet defining a coil pattern. The layers may be secured with adhesive tape, and the resonant sensor is shown in contact with the cell culture media.

FIG. 3 is a top-down view of the resonant sensor, illustrating the coil pattern defined by the resonator sheet. The PET sheet beneath the membrane transduction layer contains channels or grooves with variable kerf sizes, which modulate the sensor's response to cell growth.

FIG. 4 illustrates four examples of different kerf sizes in the channels of the PET sheet, including kerfs of 0.25 mm, 0.50 mm, 0.75 mm, and 1.00 mm. The figure demonstrates how variations in kerf size can alter sensor gain and responsiveness to cell growth phases.

FIG. 5 is a graph showing the sensor response (Skroot Growth Index or SGI) over time for different kerf sizes when monitoring the growth of E. coli in a well-mixed system with terrific broth. The graph illustrates the effect of kerf size on the sensor's ability to track cell growth through different growth phases, including lag, exponential, and stationary phases.

### DETAILED DESCRIPTION

The following detailed description refers to the accompanying drawings that show, by way of illustration, various embodiments of the invention. These embodiments are described in sufficient detail to enable those of ordinary skill in the art to practice these and other embodiments. Other embodiments may be utilized, and structural, logical, mechanical, and electrical changes may be made to these embodiments. The various embodiments are not necessarily mutually exclusive, as some embodiments can be combined with one or more other embodiments to form new embodiments. The following detailed description is, therefore, not to be taken in a limiting sense.

Secreted metabolites are recognized as a class of bio-process analytical technology (PAT) targets that can correlate to cell conditions. Chan, Y. J., Dileep, D., Rothstein, S. M., Cochran, E. W., & Reuel, N. F. (2024). Single-Use, Metabolite Absorbing, Resonant Transducer (SMART) Culture Vessels for Label-Free, Continuous Cell Progression Monitoring. Advanced Science, 11 (32), discloses an approach for a continuous metabolite monitoring strategy using a single-use metabolite absorbing resonant transducer (SMART) to correlate with cell growth. Polyacrylate is shown to absorb secreted metabolites from living cells containing hydroxyl and alkenyl groups such as terpenoids, that act as a plasticizer. Upon softening, the polyacrylate irreversibly conformed into engineered voids above a resonant sensor, changing the local permittivity which is interrogated, contact-free, with a vector network analyzer or other device. Compared to sensing using the intrinsic permittivity of cells, the SMART approach yields a 20-fold improvement in sensitivity. However, there are problems with such an approach including with sensor response and sensor sensitivity.

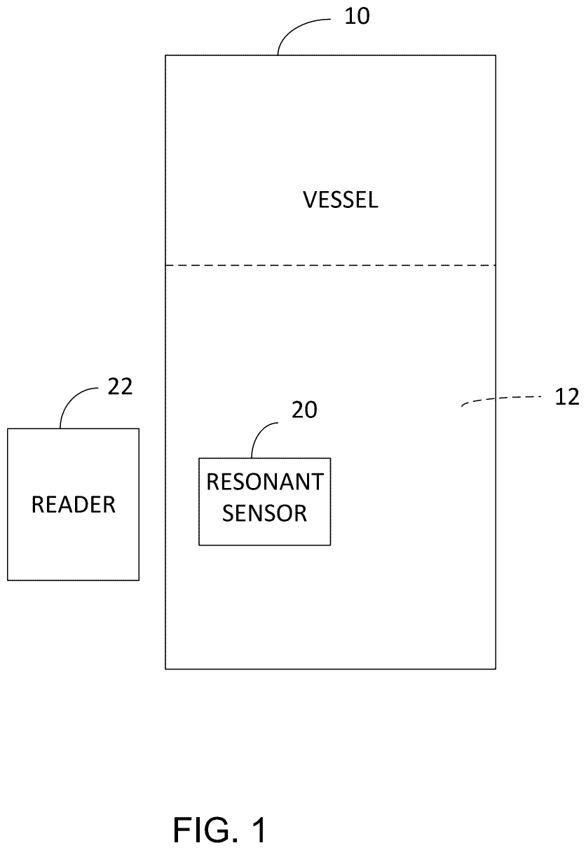

FIG. 1 illustrates an example of an environment in which the resonant sensor may be used within a cell culture device. As shown in FIG. 1, there is a vessel 10 in which a cell culture is performed with media 12. The vessel 10 has an inner surface and a resonant sensor 20 may be positioned against the inner surface of the vessel 10. For example, the resonant sensor 20 may be adhered to the inner surface of the vessel 10. The resonant sensor 20 may be a single-use, metabolite absorbing, resonant transducer. Although a single resonant sensor 20 is shown, it is contemplated that multiple resonant sensors may be used positioned at different locations within the vessel 10. Outside of the vessel 10 is a reader 22 which may be positioned proximate the resonant sensor 20 or have an antenna positioned proximate the resonant sensor 20 such that the reader 22 is in operative communication with the resonant sensor 20.

The resonant sensor 20 which may be a passive LC sensor, may be wirelessly interrogated through inductive coupling with an antenna of the reader 22. The reader may be a vector network analyzer or other device which sweeps through a range of frequencies and monitors changes in reflected or transmitted power. The resonant sensor 20 includes a sensor coil with an intrinsic inductance and parasitic capacitance (LC), which upon inductive coupling, oscillates at a specific resonant frequency.

Although the reader 22 is shown in close proximity to the resonant sensor 20, it is to be understood that the antenna of the reader 22 may be in close proximity to the resonant sensor 20 while other portions of the reader 22 may be located further away.

In some systems, multiple resonant sensors 20 may be present within the vessel 10 at different locations within the vessel 10. It should be understood that the cell culture device or vessel 10 may be of any number of volumes as the resonant sensor 20 and cell culture monitoring of the present disclosure are appropriate for both smaller volumes such as may be associated with research and development or personalized cell therapy as well as larger volumes as may be appropriate in commercial production environments. It should also be understood that the vessel 10 may be comprised of any number of different materials and need not be formed of glass or plastic. However, the material for the vessel should permit operative communication between the resonant sensor 20 and the antenna of the reader 22 and not interfere with the measurements.

It is to be understood that the reader 22 may include or be in operative communication with one or more computing devices which may include a memory and machine readable instructions for performing functions associated with operation of the resonant sensor 20 including functions associated with monitoring a cell culture, determining a particular state of the cell culture, determining a particular phase of the cell culture, predicting when a particular phase of the cell culture occur, generating an alert whenever measured or operational parameters exceed a threshold, or otherwise performing functions or operations which assist in monitoring a cell culture or operation of the system.

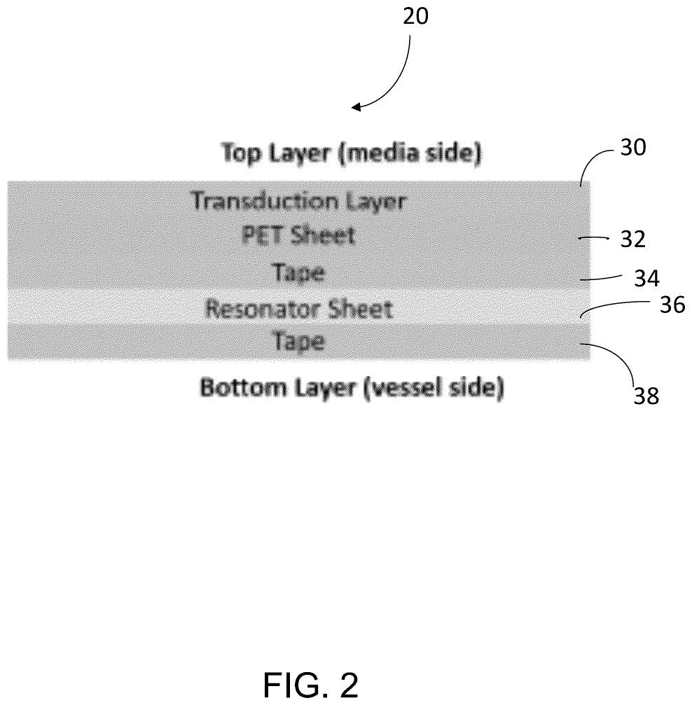

FIG. 2 is a cross-section of an example of the resonant sensor 20. The resonant sensor 20 has a top side for contact with media within a vessel and an opposite bottom side for contact with the vessel. The resonant sensor 20 may be formed of a plurality of layers of stacked materials. At the top side of the resonant sensor 20 is a cell transduction membrane 30. The cell transduction membrane 30 overlays a biocompatible sheet such as a plastic sheet such as a polyethylene terephthalate (PET) sheet 32 and a resonator sheet 36. Tape 34, 38 is shown as one method of securing layers to one another and/or the resonant sensor 20 to the inner wall or surface of a vessel. The tape 34, 38 may be an acrylic adhesive transfer tape.

The cell transduction membrane 30 may be in the form of a polyacrylate adhesive film. This polyacrylate film or other material serves as a signal-enhancing material that absorbs secreted metabolites from the cells, such as terpenoids. Upon absorbing these metabolites, the polyacrylate softens and conforms into engineered voids above the resonator sheet, changing the local permittivity.

This change in physical properties due to metabolite absorption effectively transduces cell growth into a measurable electrical signal. The change in permittivity is detected at the resonator sheet, which alters the resonant frequency. This process of converting metabolite absorption into a detectable signal results in the polyacrylate film being a transduction layer in the sensor system. Although polyacrylate is used as an example herein, it is to be understood that other types of materials may be used instead to form the cell transduction membrane.

The biocompatible sheet such as a plastic sheet or PET sheet may be laser-cut with channels or grooves, which are then layered with the cell transduction membrane. This helps create air voids between the polyacrylate and the PET sheet. The PET sheet (or other biocompatible sheet) provides a structured surface for the cell transduction membrane 30 to conform to when it softens due to metabolite absorption. As the cell transduction membrane 30 softens in response to metabolites secreted by the cells, it sinks into the channels or grooves of the PET sheet. This conformation into the air voids created by the PET sheet shows mechanical changes in the polyacrylate or cell transduction membrane 30 that are transduced into an electrical signal by the resonant sensor. It is to be understood that any number of types of biocompatible materials may be used including various types of plastics suitable for this application.

The resonant sheet 36 may include a coil pattern comprised of copper or another conductor such as silver, gold, graphene, conductive polymer, metal alloy, or other types of materials which provide the required sensitivity.

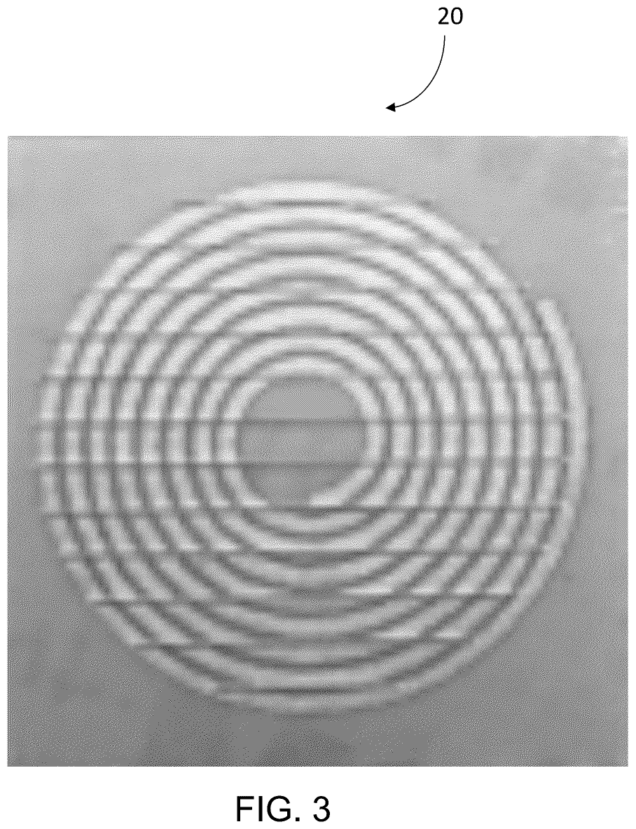

FIG. 3 is a top view of the resonant sensor 20. The resonant sensor 20 has a coil pattern defined by the resonator sheet. As shown in FIG. 3, the PET sheet has channels or grooves throughout the sheet. These channel widths referred to herein as kerf can be fabricated with different widths. The different kerfs enable altered sensor response for the resonant sensors, altering the sensor gain and how responsive the sensors are to cell growth.

The kerf may be optimized to match the diffusion and absorption behavior of the metabolites secreted by the cells. This results in optimizing the transduction layer's mechanical response and the sensors' ability to detect changes in cell growth. A larger or higher kerf would expose more surface area of the cell transduction membrane 30 to the media, allowing biomolecules to diffuse more easily into the cell transduction membrane. This may result in faster and more pronounced absorption of the biomolecules leading to a quicker and more significant softening of the transduction layer. The larger or higher kerf may provide more space for the transduction layer to deform when it softens upon absorbing metabolites thereby allowing for a more substantial change in the cell transduction membrane's conformation resulting in a more significant shift in local permittivity thereby enhancing the resonant senor's ability to detect changes.

The size of the kerf also impacts the ability to detect different growth phases. During the exponential growth phase, cells are actively dividing and secreting metabolites at a high rate. If the kerf is too large, then the polyacrylate might deform too easily and quickly such that later on there is no longer significant mechanical changes in response to more metabolites. Thus, the later more gradual changes at the end of the exponential growth phase and the transition to stationary growth may not be detected.

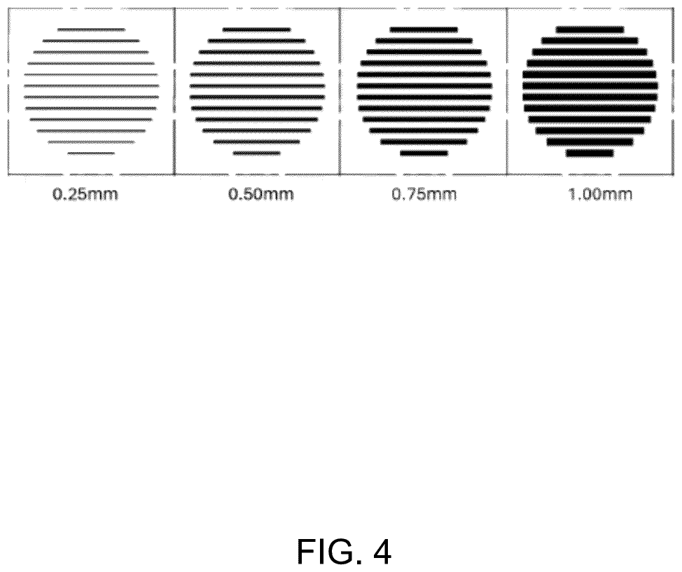

FIG. 4 illustrates four examples of different kerfs which may be used including kerfs of 0.25 mm, 0.50 mm, 0.75 mm, and 1.00 mm. The different kerfs enable altered sensor response for the resonant sensors, altering the sensor gain and how responsive the sensors are to cell growth. The particular kerf used need not be any of these specific kerfs and need not be in the range of 0.25 mm to 1.00 mm. For example, smaller kerf can be used including kerf of 0.15 mm and thus kerf may be in the range of 0.15 to 1.00 mm. The specific kerf selected may be based on the desired properties of sensor gain in a given application and thus may vary on the cells used, the metabolites measured, the type of material used in the cell transduction membrane, and other considerations as may be appropriate for the particular application. One way to select an optimized kerf for a given application is to perform measurements in the same conditions but with varying kerf sizes.

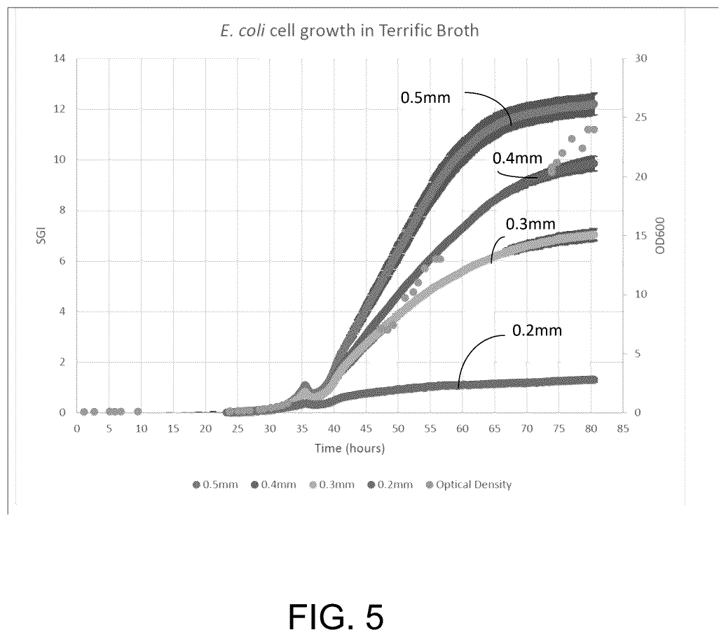

For example, the change in sensor gain with different kerf sizes is evident when monitoring the growth if E. coli in a well-mixed system with terrific broth. This is shown in FIG. 5 which includes a graph of sensor response (SGI [Skroot Growth Index]) changing as the E. coli culture grows. The growth of the E. coli culture was evaluated with periodic media sampling and subsequent spectrophotometer scans (OD600). As observed, changing the kerf in the PET channels modulates sensor gain with respect to sampled cell growth. Furthermore, changes to the kerf can also influence how fast the sensor responds to cell growth as the cells progress from one cell growth phase to another (from lag phase to exponential phase). Sensors that do not have an optimized kerf to match the growth rate of the cell culture will not properly capture late exponential and stationary phases of cell growth, or fail to capture exponential phase of cell growth. Different cell types (e.g., mammalian vs. microbial) grow at different rates and thus sensors can be tuned to match the specific cell type.

With a kerf suited for cell culture growth, the cell culture can be continuously monitored and an operator notified when the cell culture has entered into stationary phase, typically an indication of harvest time.

The sensor system is advantage over approaches such as biomass probes, as the sensor system may be used to evaluate cell growth in vessels with sizes ranging from large bioreactors down to small petri dishes. Additionally, the underlying readout method is different, with biomass probes relying on the polarization of live cells to change the probe reading and here, the sensor tracks secreted metabolites from growing cell populations. The sensor system also avoids periodic or intermittent sampling to avoid risks such as contamination, cell culture loss, disruptions to cell culture conditions, while also eliminating the need for considerable manual labor associated with such sampling.

The sensor system with adjustable kerf size is not burdened with the same limitations as optical density measurements. Instead of being limited to accurately tracking a portion of the entire cell growth curve, the disclosed sensor system tracks lag, exponential, and stationary cell growth phases without the need to sample the culture or dilute said sample. Additionally, the sensor system may be used in opaque cell culture vessels provided that short wave radio frequencies are not blocked.

Thus, the sensor system is advantageous in that evaluation of the cell culture growth is continuous, real-time, and does not require a technician to sample the culture. This sensor system enables thorough insight to cell culture growth regardless of cell behavior, like clumping. Moreover, by optimizing kerf size, the sensor system provides for a resonant sensor which is configured to capture exponential phase of cell growth including late exponential cell growth and further capture stationary phases of cell growth for the cell cultures.

Therefore, various apparatus, methods, and systems have been disclosed. The invention is not to be limited to the particular embodiments described herein. In particular, the invention contemplates numerous variations in the size, shape, geometry, and configuration of different elements, the functionality provided, the correlations between signal parameter data and physical parameters, the information presented on a display where present, and other options, variations, and alternatives. The foregoing description has been presented for purposes of illustration and description. It is not intended to be an exhaustive list or limiting to the precise forms disclosed. It is contemplated that other alternatives or exemplary aspects are considered a part of this disclosure. The description is merely examples of embodiments, processes, or methods of the invention. It is understood that any other modifications, substitutions, and/or additions can be made, which are within the intended spirit and scope of the invention. Although specific embodiments have been illustrated and described herein, it will be appreciated by those of ordinary skill in the art that any arrangement to achieve the same purpose may be substituted for the specific embodiments shown. Various embodiments use permutations and/or combinations of embodiments described herein. It is to be understood that the above description is intended to be illustrative, and not restrictive, and that the phraseology or terminology employed herein is for the purpose of description.

What is claimed:

1. An apparatus for monitoring cell cultures, comprising:
a resonant sensor comprising:
a cell transduction membrane for contact with cell culture media,
a biocompatible sheet overlaid by the cell transduction membrane,
a resonant sheet defining a coil pattern, the resonant sheet overlayed by the biocompatible sheet,
a plurality of channels formed in the biocompatible sheet, the plurality channels of having a kerf, and
wherein the kerf for the plurality of channels is selected such that the resonant sensor is configured to capture exponential phase of cell growth including late exponential cell growth and further capture stationary phases of cell growth for the cell cultures.

2. The apparatus of claim 1 wherein the resonant sensor is a single-use, metabolite absorbing, resonant sensor.

3. The apparatus of claim 1 further comprising a reader antenna in operative communication with the resonant sensor when the resonant sensor is positioned on an inner wall of a vessel during a cell culture.

4. The apparatus of claim 1 wherein the kerf is whin a range of 0.15 mm to 1.00 mm.

5. The apparatus of claim 1 wherein the cell transduction membrane comprises a polyacrylate adhesive film.

6. The apparatus of claim 1 wherein the biocompatible sheet is a polyethylene terephthalate (PET) sheet.

7. The apparatus of claim 1 wherein the coil pattern comprises copper.

8. The apparatus of claim 1 wherein the plurality of channels extend at least fully across the coil pattern.

9. A system comprising a vessel with the cell culture media and wherein the apparatus of claim 1 is secured to an inner wall of the vessel.

10. A method of monitoring cell cultures, the method comprising:
selecting a resonant sensor, the resonant sensor comprising:
a cell transduction membrane for contact with cell culture media,
a biocompatible sheet overlaid by the cell transduction membrane,
a resonant sheet defining a coil pattern, the resonant sheet overlayed by the biocompatible sheet,
a plurality of channels formed in the biocompatible sheet, the plurality channels of having a kerf, and
wherein the kerf for the plurality of channels is selected such that the resonant sensor is configured to capture exponential phase of cell growth including late exponential cell growth and further capture stationary phases of cell growth for the cell cultures;
securing the resonant sensor to an inner wall of a vessel containing the cell culture media; and
during the cell culture, monitoring cell growth using the resonant sensor.

11. The method of claim 10 wherein the resonant sensor is a single-use, metabolite absorbing, resonant sensor.

12. The method of claim 10 further comprising positioning a reader antenna in operative communication with the resonant sensor where the resonant sensor is positioned on the inner wall of a vessel during the cell culture.

13. The method of claim 10 wherein the kerf is within a range of 0.15 mm to 1.00 mm.

14. The method of claim 10 wherein the cell transduction membrane comprises a polyacrylate adhesive film.

15. The method of claim 10 wherein the biocompatible sheet is a polyethylene terephthalate (PET) sheet.

16. The method of claim 10 wherein the coil pattern comprises copper.

17. The method of claim 10 wherein the plurality of channels extend at least fully across the coil pattern.

18. A method comprising:
providing a resonant sensor, the resonant sensor comprising:
a cell transduction membrane for contact with cell culture media,
a biocompatible sheet overlaid by the cell transduction membrane,
a resonant sheet defining a coil pattern, the resonant sheet overlayed by the biocompatible sheet,
a plurality of channels formed in the biocompatible sheet, the plurality channels of having a kerf; and
tuning a dynamic response of the resonant sensor to a specific cell growth rate by adjusting size of the kerf.
